(19) United States
(12) Patent Application Publication
(10) Pub. No.: US 2012/0122830 A1
(43) Pub. Date: May 17, 2012

(54) PREGNENOLONE SULFATE FOR THE TREATMENT OF NEUROLOGIC DISORDERS

(75) Inventor: Chaker N. Adra, Boston, MA (US)

(73) Assignee: King Faisal Specialist Hospital & Research Centre, Riyadh (SA)

(21) Appl. No.: 13/157,147

(22) Filed: Jun. 9, 2011

Related U.S. Application Data
(63) Continuation of application No. PCT/US2009/006473, filed on Dec. 9, 2009.
(60) Provisional application No. 61/201,275, filed on Dec. 9, 2008.

(51) Int. Cl. A61K 31/57; C12N 5/0793; A61P 25/14; A61P 25/16; A61P 25/00; A61P 25/28

(52) U.S. Cl. 514/182; 435/375

(57) ABSTRACT

The disclosure relates, in part, to methods of using pregnenolone sulfate (PREGS) to protect against the neurotoxicity of the β-amyloid peptide Aβ₁₋₄₂. The disclosure also provides methods of using pegnanolone sulfate for treating neurologic disease or dysfunction. The disclosure further provides of using pegnanolone sulfate methods for stimulating or promoting growth of a neuronal cell.

### RELATED APPLICATIONS

This application is a continuation of PCT/US2009/006473, filed on Dec. 9, 2009, and claims the benefit of U.S. provisional application Ser. No. 61/201,275, filed on Dec. 9, 2008, and entitled “Pregnenolone Sulfate For The Treatment Of Neurologic Disorders,” the entire disclosures of both of which are incorporated herein by reference.

### FIELD OF THE INVENTION

This disclosure relates to the use of pregnenolone sulfate, a neuroactive steroid, to prevent and treat neurologic disease or dysfunction.

### BACKGROUND OF THE INVENTION

Neurodegenerative diseases are characterized by a chronic, progressive degeneration of neurons resulting in nervous system dysfunction and often eventually lead to death. According to the National Institute of Neurological Disorders and Stroke, neurodegenrative diseases encompass more than 600 neurological disorders and affect about 50 million Americans each year. Billions of dollars are spent each year in the United States on direct health care costs and lost opportunities with Alzheimer's disease alone costing an estimated 100 billion dollars every year (Brown et al., (2005) Environmental Health Perspectives).

Neurodegenerative diseases such as Alzheimer's disease, Down's syndrome and Lewy body dementia have been associated with deposition of the amyloid plaques in neuronal tissue. These plaques are composed of a tangle of fibrillar aggregates composed of β-amyloid peptides (Aβ). The most common isoforms are Aβ₁₋₄₀ and Aβ₁₋₄₂ although Aβ₁₋₄₂ is more fibrillogenic and associated with disease states.

Currently, there are few therapies, if any, for the varied range of neurological diseases. Often, the available treatments offer minimal symptomatic benefits that concentrate solely on reducing the severity or intensity of disease symptoms. It would be beneficial to have additional approaches that aim to prevent and/or treat the deleterious effects of neurodegenerative diseases.

### SUMMARY OF THE INVENTION

The disclosure is based, in part, on the discovery that pregnenolone sulfate (PREGS) shows neurotrophic activity and provides neuroprotection against β-amyloid peptide-induced neurotoxicity.

According to one aspect of the disclosure a method for treating a subject having a neurologic disease or dysfunction is provided. The method comprises administering to the subject pregnenolone sulfate in an amount effective to treat neurologic disease or dysfunction. Non-limiting examples of neurologic disease or dysfunction include age-related neurodegeneration, Alzheimer's disease, Amyotrophic lateral sclerosis, Alper's diseases, Batten disease, Bovine spongiform encephalopathy (BSE), chemotherapy-induced neuropathy, Creutzfeldt-Jakob disease, diabetic neuropathy, Down's syndrome, frontotemporal lobar degeneration, Huntington's disease, HIV-associated dementia, Krabbe's disease, Lewy body dementia, multiple system atrophy, Niemann Pick disease, Parkinson's disease, Polyneuritis, Prion disease, Primary lateral sclerosis, Refsum's disease, Sandhoff s disease, senile dementia, Spielmeyer-Vogt-Sjogren-Batten disease, Spinocerebellar ataxia, Spinal muscular atrophy, Subacute combined degeneration of spinal cord, Tabes dorsalis, or Vascular dementia.

In some embodiments, the neurologic disease or dysfunction is caused by a burn, traumatic injury, mechanical injury, surgical injury, physiological injury, pathological injury or immunological injury. In some embodiments, the neurologic disease or dysfunction is a neurodegenerative disease. The neurodegenerative disease may be caused by the accumulation of β-amyloid peptide such as Aβ_{1-42.}

According to another aspect of the disclosure, a method for treating a subject having a condition associated with β-amyloid peptide accumulation in neuronal tissue is provided. The method comprises administering to the subject pregnenolone sulfate in an amount effective to treat the condition. In some embodiments, the condition is Alzheimer's disease, Down's syndrome or Lewy body dementia.

According to still another aspect of the disclosure, a method for reducing or preventing the accumulation of β-amyloid peptides in neuronal tissue is provided. The method comprises contacting the neuronal tissue with pregnenolone sulfate. In some embodiments, the β-amyloid peptide is Aβ_{1-42.}

According to another aspect of the disclosure, a method for stimulating or promoting growth of a neuronal cell is provided. The method comprises contacting the neuronal cell with pregnenolone sulfate in an amount effective to stimulate or promote neuronal cell growth. In some embodiments, the neuronal cell is in a tissue. The tissue may be brain tissue, spinal cord tissue or peripheral nervous tissue. In some embodiments, the neuronal growth comprises neuronal outgrowth.

The following embodiments apply equally to the above aspects of the disclosure set forth herein unless indicated otherwise.

In some embodiments, the pregnenolone sulfate used is the synthetic (−) enantiomer of PREGS (ent-PREGS). In other embodiments, the pregnenolone sulfate is administered orally, intravenously, intramuscularly, intrathecally, sublingually, buccally, intranasally, intra-articularly, intraperitoneally, subcutaneously, or topically. In some embodiments, the pregnenolone sulfate is administered prophylactically.

In some embodiments, the subject is otherwise free of indications calling for treatment with pregnenolone sulfate. In other embodiments, agents other than pregnenolone sulfate are also administered to the subject.

Each of the limitations of the disclosure can encompass various embodiments of the disclosure. It is, therefore, anticipated that each of the limitations of the disclosure involving any one element or combinations of elements can be included in each aspect of the disclosure. The disclosure is capable of other embodiments and of being practiced or of being carried out in various ways. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

These and other aspects of the disclosure, as well as various advantages and utilities will be apparent with reference to the Detailed Description. Each aspect of the disclosure can encompass various embodiments as will be understood. All documents identified in this application are incorporated in their entirety herein by reference

### BRIEF DESCRIPTION OF THE DRAWINGS

The accompanying drawings are not intended to be drawn to scale. For purposes of clarity, not every component may be labeled in every drawing. In the drawings:

FIG. 1 is a graph showing the effect of PREGS on B104 cell viability. B104 cells treated with PREGS during 24 h at various concentrations ranging from 0.25 to 40 μM. Results are expressed in percentage of cell viability and are average of triplicate experiments±standard error. PREGS is not toxic and does not affect the viability of the B104 cells.

FIG. 2 are photographs showing the neurotrophic activity of PREGS. Neuronal outgrowth in B104 cells cultured at low density (A) In absence of PREGS, (B) in presence of 5 μM PREGS during 7 days. Arrows show a striking outgrowth induced by PREGS.

FIG. 3 is a histogram showing the necrotic effect of the fibrillary form of human Aβ₁₋₄₂ peptide (fAβ1-42) on B104 cells. The necrotic effect was demonstrated as early as 6H after treatment. A significant dose-dependent necrosis was observed increasing from 5 μM to 20 μM of fAβ1-42 peptide. The percentages of necrotic cells are average of duplicate experiments±standard error.

FIG. 4 are histograms showing the neuroprotection by PREGS against the fibrillary form of human Aβ₁₋₄₂ peptide (fAβ1-42) toxicity on B104 cells. PREGS at 20 μM was able to protect B104 cell against toxicity induced by 20 μM of fAβ1-42 peptide. The effect was observed at 24H and maintained until 96H.

### DETAILED DESCRIPTION OF THE INVENTION

The disclosure described herein relates, in part, to pregnenolone sulfate (PREGS) and methods of using PREGS in the treatment and prevention of neurological disease or dysfunction. The disclosure also relates to the ability of PREGS to protect against the neurotoxicity and neurodegeneration caused by the accumulation of β-amyloid peptides in neuronal tissues. In some embodiments, the methods of the disclosure are directed to the use of PREGS as a neurotrophic agent to stimulate or promote neuronal cell growth. In some embodiments, the pregnenolone sulfate used is the synthetic (−) enantiomer of PREGS (ent-PREGS).

Methods of the disclosure comprise administering an effective amount of prenenolone sulfate to a subject in need thereof to treat, prevent or ameliorate neurologic disease or dysfunction. PREGS belongs to the group of neurosteroids that can affect neuronal excitability through interaction with neurotransmitter-gated ion channels. ent-PREGS, the synthetic (−) enantiomer of PREGS, is more potent in stimulating memory in rats and mice than its physiological counterpart (Akwa et al. Proc. Nat. Acad. Sci. USA. 2001; 98:14033-37). Pregnenolone sulfate and its synthetic enantiomeric analogue ent-PREGS differ from classical steroid hormones in the chemical structure and are known to regulate brain functions, independently from the endocrine mechanisms.

The term “neurologic disease or dysfunction” as used herein refers to a nervous system disorders characterized by damage, deterioration or loss of neuronal tissue and/or neuronal tissue function. Examples of neurologic disease or dysfunction include, but are not limited to, age-related neurodegeneration, Alzheimer's disease, Amyotrophic lateral sclerosis, Alper's diseases, Batten disease, Bovine spongiform encephalopathy (BSE), chemotherapy-induced neuropathy, Creutzfeldt-Jakob disease, Down's syndrome, diabetic neuropathy, frontotemporal lobar degeneration, Huntington's disease, HIV-associated dementia, Krabbe's disease, Lewy body dementia, multiple system atrophy, Niemann Pick disease, Parkinson's disease, Polyneuritis, Prion disease, primary lateral sclerosis, Refsum's disease, Sandhoff s disease, senile dementia, Spielmeyer-Vogt-Sjogren-Batten disease, Spinocerebellar ataxia, spinal muscular atrophy, subacute combined degeneration of spinal cord, Tabes dorsalis or vascular dementia. In some embodiments, the neurologic disease or dysfunction is caused by burn, traumatic injury, mechanical injury, surgical injury, physiological injury, pathological injury or immunological injury. In some embodiments, the neuronal cell injury or dysfunction do not include Alzheimer's disease and age-related dementia.

The term “disease” or “disorder” is used interchangeably herein, and refers to any modification in state of the body or of some of the organs that interrupts or disturbs bodily functions and/or causes symptoms such as discomfort, dysfunction, distress, or even death to the subject afflicted.

“Neurodegenerative diseases” are characterized by the gradual and progressive loss of neuronal tissue and/or neuronal tissue function. In some embodiments, the neurodegenerative disease is characterized by the accumulation of β-amyloid peptides (Aβ). Aβ peptides of 39-43 amino acid residues are formed by the sequential cleavage of the amyloid precursor protein by α, β and γ-secretases. The Aβ₁₋₄₂ peptide is the more fibrillogenic and is believed to be a major component of amyloid plaques that appear to contribute to the deterioration of neurons and subsequent development of disease states. Accordingly, on aspect the present disclosure is directed to treating the accumulation of β-amyloid peptides in neuronal tissues such as brain, spinal cord and the peripheral nervous system. Further, the present disclosure is also directed to treating, preventing or ameliorating conditions characterized by the accumulation of β-amyloid peptides in neuronal tissues. Examples of such conditions include, but are not restricted to, Alzheimer's disease, Down's syndrome or Lewy body dementia. The rate of apoptosis or necrosis that reflects the neurotoxic effect of Aβ peptide as well as its regulation by neurosteroids is studied mainly by flow cytometry and other cell survival assays. In addition, the potential neurotrophic activity of neurosteroids is analyzed by immunochemistry using specific neuronal markers.

Alzheimer disease (AD) first described by Alois Alzheimer in 1907 is a progressive neurodegenerative disease that leads to brain atrophy, cognitive deficits and dementia. It is characterized by senile plaques and neurofibrillary tangles (Hyman, Neurobiol Aging 1997, 18: 527-32; Delacourte et al., Neurology 1999, 52:1158-65; Selkoe, JAMA 2000, 283:1615-1617). The main pathological components of neurofibrillary tangles are paired helical filaments of highly phosphorylated microtubule-associated protein tau (Grundke-Iqbal et al., Proc Natl Acad Sci USA. 1986, 83: 4913-17; Buée et al., Brain Res Rev 2000, 33:95-30) that provokes destabilization of neuronal cytoskeleton leading to neurodegeneration (Alonso et al., Proc. Nat. Acad. Sci. USA. 1997, 94: 298-03). Senile plaques result mainly from the accumulation of excessive amounts of β-amyloid (Aβ) protein (Hyman, Neurobiol Aging 1997, 18: 527-32; Selkoe, JAMA 2000, 283:1615-1617). This Aβ protein is normally produced as a soluble metabolic product of amyloid precursor protein and can be detected as circulating peptide in the plasma and CSF of healthy humans (Haass et al., Nature 1992, 359:322-35; Selkoe, Physiol Rev 2001, 81:741-66). The brain aggregation of Aβ peptides, particularly of Aβ₁₋₄₂, is known to be highly toxic to neurons in vivo and in vitro (LeFerla et al. Nat Genet 1995, 9(1):21-30; Tan et al. Journal of Neurochemistry 1998, 71: 95-105; Shimohama et al. Apoptosis 2000, 5(1): 21-30).

AD is so far of unknown etiology. Epidemiological studies indicate that AD is the principal cause of dementia in aged people over 65 years (Helmer et al., Med Sci (Paris). 2006, 22: 288-96). Its prevalence is estimated to be about 50 to 100 millions cases worldwide. Because of the increase of life expectancy and aging of the population, AD is becoming a social and economical public health problem to urgently overcome.

Unfortunately, there is currently no cure for AD. The only available medications (cholinesterase inhibitors and NMDA receptor ligands) offer relatively small symptomatic benefit for some patients, but do not slow disease progression (Desai and Gossberg, Neurology 2005, 64:S34-39).

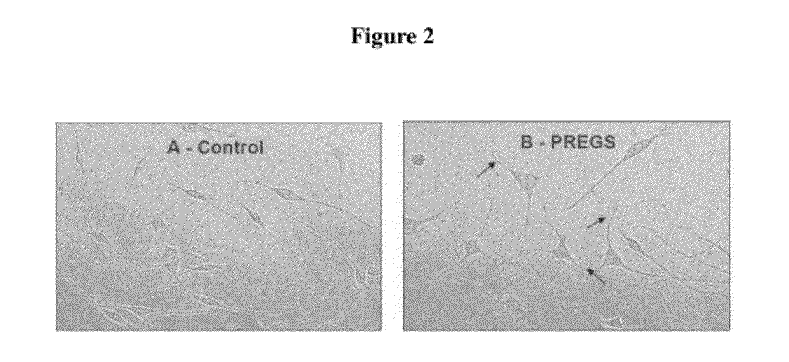

The central nervous system (CNS) has the capacity to produce its proper steroids called “neurosteroids” (Baulieu et al., Biol Cell 1991, 71: 3-10; Schumacher et al., Prog Neurobiol 2003, 71:3-29), and the enzymes implicated in their biosynthesis and metabolism from cholesterol have been identified in neurons and glial cells (FIG. 2; Akwa et al., J Steroid Biochem Mol. Biol. 1991, 40:71-81; J. Cell. Biol. 1993, 121:135-43; Zwain et al., Endocrinology 1999, 140: 3843-52; Mensah-Nyagan et al., Pharmacol Rev 1999, 51:63-81; Mellon et al., Trends Endocrinol Metab 2002, 13(1):35-43). Many steroids are known to influence CNS activity, and among these neuroactive steroids are neurosteroids that regulate different brain functions, such as memory, response to stress, anxiety or sleep, as shown by psychopharmacological and behavioral studies in animal models (Akwa et al., J Soc Biol 1999, 193:293-98; Rupprecht et al., Int Rev Neurobiol 2001, 46: 461-77). In particular, pregnenolone (PREG) and its sulfate derivative PREGS are able to enhance memory performance in young adult rodents (Flood et al., Proc Natl Acad Sci USA 1992, 89: 1567-71; Proc Natl Acad Sci USA 1995, 92: 10806-10; Darnaudery et al., Brain Res 2000, 852:173-79; Akwa et al., Proc Nat Acad Sci USA 2001, 98:14033-37) and to reverse amnesia caused by pharmacological agents (Meziane et al., Psychopharmacology (Berl) 1996, 126: 323-30; Maurice et al., Neuroscience 1998, 3:413-28), related to aging (Vallée et al., Proc Natl Acad Sci USA 1997, 94: 14865-70) or induced by Aβ peptides (Maurice et al., Neuroscience 1998 3:413-28). It is noteworthy that the synthetic enantiomer of PREGS (ent-PREGS) also stimulates memory and is more potent than the natural PREGS (Akwa et al., Proc Nat Acad Sci USA 2001, 98:14033-37).

Besides their promnesiant and anti-amnesiant properties, neurosteroids can play a role in neuroprotection (Schumacher et al., J Neurocytol 2000, 29: 307-26). PREG reduces the death caused by amyloid peptides in cultures of hippocampal HT-22 neurons (Gursoy et al., Neurochem Res 2001, 26(1):15-21). Progesterone (PROG) protects neurons after brain contusion injury or cerebral ischemia (Roof et al., Exp Neurol 1994, 129: 64-9; Gonzales-Vidal et al., Arch Med Res 1998, 29:117-24), and against Aβ protein neurotoxicity (Goodman et al., J Neurochem 1996, 66: 1836-44). Neurotrophic and neuroprotective effects of 17β-estradiol (E₂) are also well documented (Fereira et al., J Neurosci 1991, 11:392-400; Murphy et al., J Neurosci 1996, 16:4059-68; Goodman et al., J Neurochem 1996, 66: 1836-44). Neuroprotection by E₂ against amyloid peptide neurotoxicity is described (Goodman et al., J Neurochem 1996, 66: 1836-44; Green et al., J Neurocytol 2000, 29: 419-23) as well as that of its nonfeminizing enantiomer derivative (Green et al., Endocrinology 2001, 142:400-06). In this context, the effects of PREGS and ent-PREGS against Aβ peptide induced neuronal toxicity remains to be evaluated.

In humans, the knowledge on neurosteroid activity is still limited. Nevertheless, the presence of several neurosteroids has been revealed in human brain tissue (Lanthier et al., J Steroid Biochem 1986, 25: 445-49; Lacroix et al., J Steroid Biochem 1987, 28: 317-25; Weill-Engerer et al., J. Clin Endocrinol Metab 2002, 87: 5138-43), as well as the enzymes involved in their biosynthesis and metabolism (Stoffel-Wagner, Eur J Endocrinol 2001, 145: 669-679; Weill-Engerer et al., Brain Res 2003, 969:117-25; Yau et al, Neuroscience 2003, 121: 307-14). Interestingly, several brain regions from aged patients with AD, compared to non-demented subjects, contain significantly low concentrations of certain neurosteroids that they are inversely correlated with high levels of Aβ protein and hyperphosphorylated tau protein (Weill-Engerer et al., J Clin Endocrinol Metab 2002, 87: 5138-43). These findings suggest that neurosteroids may protect neurons against the damage caused by senile plaques and neurofibrillary tangles (Schumacher et al., Prog Neurobiol 2003, 71:3-29) and highlights the importance of the potential role of such steroids in AD (Akwa et al, Alzheimer Dis Assoc Disord 2005, 19: 226-39).

As used herein, a “subject in need thereof” is for example, a human who has currently or has previously had a neurologic disease or dysfunction. In addition, a subject may be suspected of having a neurologic disease or dysfunction or may be considered by one of skill in the medical arts to be at risk or at an elevated risk of having a neurologic disease or dysfunction. The neurologic disease or dysfunction may be caused by burn, traumatic injury, mechanical injury, surgical injury, physiological injury, pathological injury or immunological injury. In some aspects, the neurologic disease may be caused by neurodegenerative disease that may be characterized by the accumulation of β-amyloid peptides such as Aβ₁₋₄₂ peptide.

In some embodiments, the subject is otherwise free of indications calling for treatment with pregnenolone sulfate. A subject free of indications calling for treatment with pregnenolone sulfate is a subject who has no signs or symptoms calling for treatment with pregnenolone sulfate. Indications calling for treatment with pregnenolone sulfate are known to those of ordinary skill in the art. Examples of such indications include amnesia, premenstrual syndrome, sleeping problems and depression.

The term “treating” or “treat” is intended to include prophylaxis, amelioration, prevention or cure of a condition. Treatment after a condition has started aims to reduce, ameliorate or altogether eliminate the condition, and/or one or more of its associated symptoms, or prevent it from becoming worse. Treatment of subjects before a condition has started (i.e., prophylactic treatment) aims to reduce the risk of developing the condition and/or lessen its severity if the condition later develops. As used herein, the term “prevent” refers to the prophylactic treatment of subjects who are at risk of developing a condition which treatment results in a decrease in the probability that the subject will develop the condition, or results in an increase in the probability that the condition is less severe than it would have been absent the treatment. A response to a treatment method of the disclosure can, for example, be measured by determining the physiological effects of the treatment, such as the decrease or lack of symptoms following administration of the treatment.

In some aspects, the methods of the disclosure may help stimulate or promote growth of a neuronal cell(s). By “stimulating or promoting growth of neuronal cell” it is intended to include induction of survival, differentiation and growth of the neuronal cell. In some embodiments, the neuronal cell is in a tissue. The tissue may be brain tissue, spinal cord tissue or the peripheral nerve tissue.

In some embodiments, the neuronal growth comprises neuronal outgrowth. Neuronal outgrowth involves the growth of the axon towards the target cells with which it can form synapses. The establishment of such patterns of neuronal connectivity are essential for the proper functioning of the nervous system. This property can be assessed using a variety of assays known in the art. For instance, a nerve can be directly examined using immunofluorescence in the presence or absence of the treatment. A nerve which has longer neurites (including axons and/or dendrites) in the presence of the treatment compared to “prior to treatment” or compared to a control is one in which nerve cell growth has been enhanced. The nerve cells may be treated in vivo, in vitro, or ex vivo. Thus, the cells may be in an intact subject or isolated from a subject or alternatively may be an in vitro cell line.

An “amount effective” of pregnolone sulfate is the amount necessary or sufficient to provide a medically desirable biological result in a subject (e.g., to treat, prevent or ameliorate the neurologic disease or dysfunction). Alternatively, the desirable biological effect may include stimulating the outgrowth of neuronal cells. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. This amount can be determined empirically using known methods and will vary from subject-to-subject. It is generally preferred that a maximum dose of the pharmacological agents of the disclosure (alone or in combination with other therapeutic agents) be used, that is, the highest safe dose according to sound medical judgment. Generally, doses of pregnenolone sulfate would be from about 10-100 mg. Lower doses will result from other forms of administration, such as intravenous administration. In the event that a response in a subject is insufficient at the initial doses applied, higher doses (or effectively higher doses by a different, more localized delivery route) may be employed to the extent that subject's tolerance permits.

It should be understood that pregnenolone sulfate is used to treat or prevent neurologic disease or dysfunction, that is, it may be used prophylactically in subjects at risk of developing neuronal cell injury or dysfunction. Thus, an effective amount is that amount which can lower the risk of, delay the onset or perhaps prevent altogether the development of neurologic disease or dysfunction. It will be recognized when the pregnenolone sulfate is used in acute circumstances, it is used to prevent one or more medically undesirable results that typically flow from such adverse events.

Pregnenolone sulfate may be combined with additional therapeutic agents, such as those used in the treatment of neurologic disease or dysfunction. Examples of other therapeutic agents that may be used include, but are not limited to acetylcholinesterase inhibitors (e.g., donepezil, galantamine and rivastigmine), NMDA receptor antagonist (e.g., memantine), dopamine agonists (e.g., bromocriptine, pergolide) and monoamine oxidase-B inhibitors (e.g., selegiline and rasagiline).

Pregnenolone sulfate and other therapeutic agent(s) may be administered simultaneously or sequentially. When the other therapeutic agents are administered simultaneously they can be administered in the same or separate formulations, but are administered at the same time. The administration of the other therapeutic agents and the pregnenolone sulfate may also be temporally separated, meaning that the therapeutic agents are administered at a different time, either before or after, the administration of the pregnenolone sulfate. The separation in time between the administration of these compounds may be a matter of minutes or it may be longer.

When administered, pregnenolone sulfate and other therapeutic agent(s) are preferably administered as pharmaceutical preparations applied in pharmaceutically-acceptable amounts and in pharmaceutically-acceptably compositions. Such preparations may contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the disclosure. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Pregnenolone sulfate may be combined, optionally, with a pharmaceutically-acceptable carrier. The term “pharmaceutically-acceptable carrier” as used herein means one or more compatible solid or liquid filler, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride, chlorobutanol, parabens and thimerosal.

The pharmaceutical compositions may conveniently be presented in unit dosage form and may be prepared by any of the methods well known in the art of pharmacy. All methods include the step of bringing the active agent into association with a carrier, which constitutes one or more accessory ingredients. In general, the compositions are prepared by uniformly and intimately bringing the active compound into association with a liquid carrier, a finely divided solid carrier, or both, and then, if necessary, shaping the product.

In some embodiments, pregnenolone sulfate may be administered orally, intravenously, intramuscularly, intrathecally, sublingually, buccally, intranasally, intra-articularly, intraperitoneally, subcutaneously, or topically. Pregnenolone sulfate, when it is desirable to deliver it systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compound may be in powder form for constitution with a suitable vehicle (e.g., saline, buffer, or sterile pyrogen-free water) before use.

Compositions suitable for oral administration may be presented as discrete units, such as capsules, tablets, pills, lozenges, each containing a predetermined amount of the active compound (pregnenolone sulfate). Other compositions include suspensions in aqueous liquids or non-aqueous liquids such as a syrup, elixir, an emulsion, or a gel.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, sorbitol or cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also contemplated are oral dosage forms of the above. A component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of pregnenolone sulfate or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm The formulation of the material for capsule administration could also be as a powder, lightly compressed plugs or even as tablets. The therapeutic could be prepared by compression.

Colorants and flavoring agents may all be included. For example, pregnenolone sulfate may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Ho, Emdex, STA-Rx 1500, Emcompress and Avicell.

Disintegrants may be included in the formulation of the therapeutic into a solid dosage form. Materials used as disintegrants include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000. Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the pregnenolone sulfate either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner

For administration by inhalation, the compound for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of pregnenolone sulfate. Pregnenolone sulfate is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13 (suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-γ and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this disclosure are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this disclosure are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of pregnenolone sulfate. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically pregnenolone sulfate may also be prepared in different formulations depending on the type of chemical modification or the type of device employed.

Formulations suitable for use with a nebulizer, either jet or ultrasonic, will typically comprise pregnenolone sulfate dissolved in water or other pharmaceutically acceptable solvent. The formulation may also include a buffer and a simple sugar (e.g., for stabilization of the pregnenolone sulfate and regulation of osmotic pressure). The nebulizer formulation may also contain a surfactant, to reduce or prevent surface induced aggregation of the pregnenolone sulfate caused by atomization of the solution in forming the aerosol.

Formulations for use with a metered-dose inhaler device will generally comprise a finely divided powder containing pregnenolone sulfate suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing pregnenolone sulfate and may also include a bulking agent, such as lactose, sorbitol, sucrose, or mannitol in amounts which facilitate dispersal of the powder from the device, e.g., 50 to 90% by weight of the formulation. Pregnenolone sulfate should most advantageously be prepared in particulate form with an average particle size of less than 10 mm (or microns), most preferably 0.5 to 5 mm, for most effective delivery to the distal lung.

Nasal (or intranasal) delivery of a pharmaceutical composition of the present disclosure is also contemplated. Nasal delivery allows the passage of a pharmaceutical composition of the present disclosure to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present disclosure solution into a chamber of defined volume, which chamber has an aperture dimensioned to aerosolize and aerosol formulation by forming a spray when a liquid in the chamber is compressed. The chamber is compressed to administer the pharmaceutical composition of the present disclosure. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

Pregnenolone sulfate may also be formulated in rectal or vaginal compositions such as suppositories or retention enemas, e.g., containing conventional suppository bases such as cocoa butter or other glycerides.

In addition to the formulations described previously, pregnenolone sulfate may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic agent(s), including but not limited to pregnenolone sulfate, may be provided in particles. Particles as used herein means nano or micro particles (or in some instances larger) which can consist in whole or in part of pregnenolone sulfate or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain pregnenolone sulfate in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

For topical administration to the eye, nasal membranes, mucous membranes or to the skin, pregnenolone sulfate may be formulated as ointments, creams or lotions, or as a transdermal patch or intraocular insert or iontophoresis. For example, ointments and creams can be formulated with an aqueous or oily base alone or together with suitable thickening and/or gelling agents. Lotions can be formulated with an aqueous or oily base and, typically, further include one or more emulsifying agents, stabilizing agents, dispersing agents, suspending agents, thickening agents, or coloring agents. (See, e.g., U.S. Pat. No. 5,563,153, entitled “Sterile Topical Anesthetic Gel”, issued to Mueller, D., et al., for a description of a pharmaceutically acceptable gel-based topical carrier.)

In general, pregnenolone sulfate is present in a topical formulation in an amount ranging from about 0.01% to about 30.0% by weight, based upon the total weight of the composition. Preferably, pregnenolone sulfate is present in an amount ranging from about 0.5 to about 30% by weight and, most preferably, pregnenolone sulfate is present in an amount ranging from about 0.5 to about 10% by weight. In one embodiment, the compositions of the disclosure comprise a gel mixture to maximize contact with the surface of the localized pain and minimize the volume and dosage necessary to alleviate the localized pain. GELFOAM® (a methylcellulose-based gel manufactured by Upjohn Corporation) is a preferred pharmaceutically acceptable topical carrier. Other pharmaceutically acceptable carriers include iontophoresis for transdermal drug delivery.

The disclosure also contemplates the use of kits. In some aspects of the disclosure, the kit can include a pharmaceutical preparation vial, a pharmaceutical preparation diluent vial, and pregnenolone sulfate. The vial containing the diluent for the pharmaceutical preparation is optional. The diluent vial contains a diluent such as physiological saline for diluting what could be a concentrated solution or lyophilized powder of pregnenolone sulfate. The instructions can include instructions for mixing a particular amount of the diluent with a particular amount of the concentrated pharmaceutical preparation, whereby a final formulation for injection or infusion is prepared. The instructions may include instructions for treating a subject with an effective amount of pregnenolone sulfate. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this disclosure, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

The present disclosure is further illustrated by the following Example, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### PREGS Effect on B104 Cell Viability:

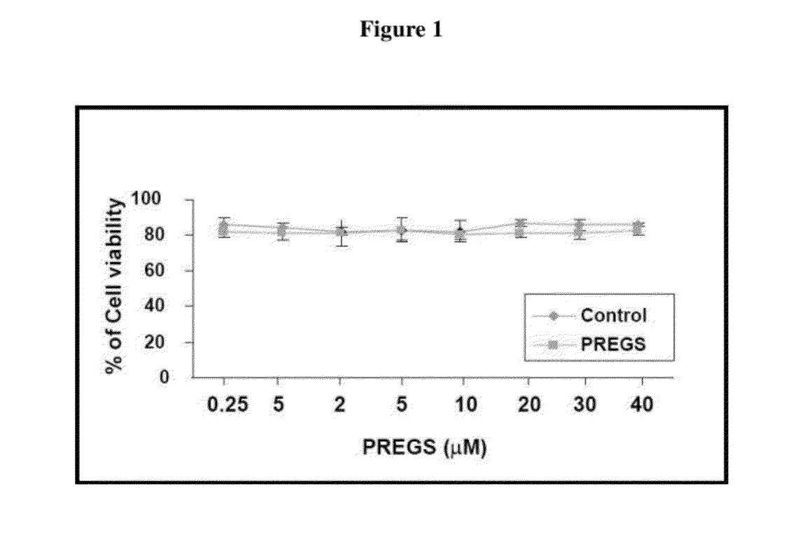

To determine if PREGS was neurotoxic, a dose-response study was conducted in cultured B104 cells. B104 cells were treated with various concentrations of PREGS, ranging from 0.25 to 40 μM, for 24 h. The results expressed in percentage of cell viability showed that PREGS is not neurotoxic and does not affect the viability of these cells as compared to control cells (FIG. 1).

### Example 2

### Neurotrophic Activity of PREGS:

The neurotrophic activity of PREGS was assayed in B104 cells cultured at low density. Striking neuronal cell outgrowth was observed in the presence of 5 μM PREGS during 7 days as compared to control cells (FIG. 2).

### Example 3

### Necrotic Effect of Fibrillary Form of Human Aβ₁₋₄₂ Peptide on B104 Cells:

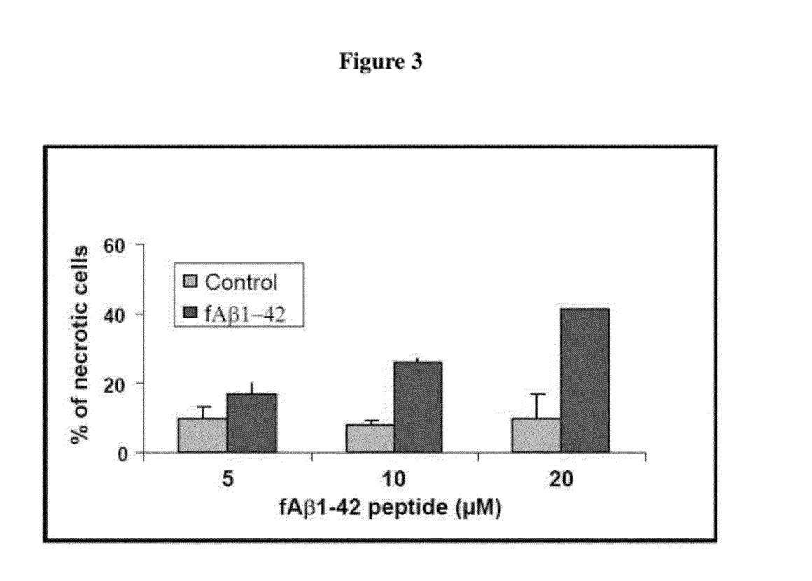

B104 cells demonstrated a considerable necrotic effect after only 6 h of treatment with 10 μM human Aβ₁₋₄₂ peptide as compared to control cells. Moreover, a significant dose-dependent necrosis was observed increasing from 5 mM to 20 mM of Aβ₁₋₄₂ peptide (FIG. 3).

### Example 4

### Neuroprotection by PREGS Against the Fibrillary Form of Human Aβ₁₋₄₂ Peptide Toxicity on B104 Cells:

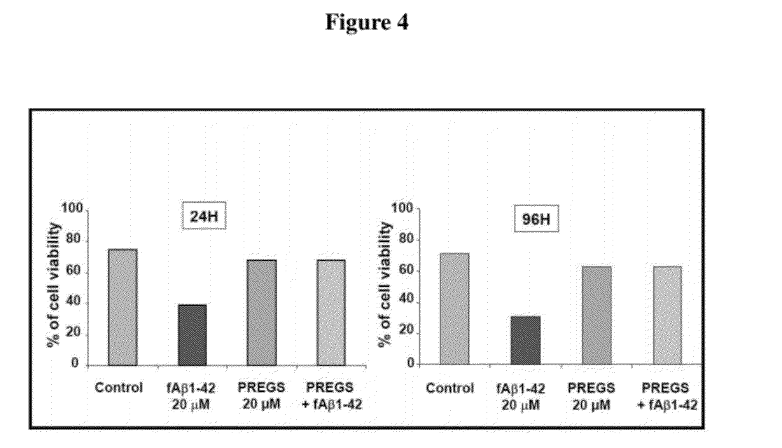

The capacity of PREGS to correct human Aβ₁₋₄₂ peptide-induced neurotoxicity was analyzed. PREGS at 20 mM was able to protect B104 cells against toxicity induced by 20 mM of human Aβ₁₋₄₂ peptide. This effect was observed at 24 h and maintained until 96 h (FIG. 4).

### Example 5

### Materials and Methods

### a) Cell Culture Model

Rat B104 neuroblastoma is chosen as the cell culture model due to its neuronal phenotype when grown in serum free medium (Schubert et al. Nature 1974; 249: 224-27). B104 cells are routinely grown in flasks of 75 cm² with complete culture medium containing Dulbecco's Modified Eagle Medium (DMEM) supplemented with 2 mM L-glutamine, penicillin G (50 U/ml), streptomycin sulfate (50 μg/ml), 10% fetal calf serum, and 5% horse serum. They are sub-cultured every week and the culture medium is changed every three days. Cultures are maintained at 37° C. in a humidified incubator in 90% air and 10% CO₂ atmosphere. For the experiments, the cells are plated at the required density, in serum complete medium for 24 H and then the medium is replaced with a serum free one containing or not the desired treatment.

### b) Neurosteroid and β-Amyloid Peptide Preparation

The steroids PREG (Sigma), PREGS (Steraloids), ent-PREGS (St. Louis, Mo.), PROG (Sigma), allopregnanolone (Sigma) and E2 (Sigma) are prepared at the initial concentration of 1M in ethanol (0.05%). A dose-response is conducted and steroids are diluted in culture medium to the final concentration required for the experiments. PROG and E2 are used as positive controls.

Aβ₁₋₄₂ is obtained from Bachem and prepared in fibril form known to mediate neurotoxicity (Lorenzo et al. Proc Natl Acad Sci USA 1994; 91:12243-47). Aβ₁₋₄₂ fibrils are produced by dissolving the peptide in double distilled water (ddH₂O) to 10 82 g/μl, followed by incubation under agitation (300-400 rpm) for one week at 37° C.

Two different and complementary approaches are explored in vitro, using B104 human neuroblastoma cells in culture: 1) the potential neurotrophic effects of PREGS or ent-PREGS and 2) the potential neuroprotective activities of PREGS or ent-PREGS, against the neurotoxicity induced by Aβ₁₋₄₂ peptide. In both types of studies, E2, PROG or steroid metabolites such as allopregnanolone (reduced metabolite of PROG) serve as positive controls, as they are known to display neuroprotective properties in different models of neuronal toxicity.

### Cell Survival- Flow Cytometry

Flow cytometry is utilized to look for the percentage of apoptosis or necrosis that reflects the neurotoxic effect of Aβ₁₋₄₂ as well as the neuroprotective effect of neurosteroids by staining cells with annexin V that has affinity to phosphatidyl serine (PS) (Van Genderen et al. 2006). In normal viable cells, PS is located on the cytoplasmic side of the cell membrane. However, in apoptotic cells, PS is translocated from the inner to the outer leaflets of the plasma membrane where annexin V could bind to PS. Moreover, annexin V can pass through the compromised membrane of necrotic cells and bind to PS in the anterior of the cell (Van Genderen et al. Nat Protoc 2006; 1:363-67).

Cells are seeded in 6 well plates at the required density and grown for 24H. They are then treated according to two procedures: either with specific steroids and then Aβ₁₋₄₂ or with Aβ₁₋₄₂ followed by steroids. A kinetic study is performed at 6, 12 or 24 H depending on the concentration of the peptide.

For flow cytometry analysis, cells are washed with PBS and harvested by trypsinization. After centrifugation and resuspension in annexin binding buffer and incubation for 15 min with 5-25 μl of annexin V conjugate, cells are washed with annexin binding buffer and examined by flow cytometry. Data is examined by using appropriate software, and scattergrams are analyzed.

### Example 6

MTT Assay

The neurotoxic effect of Aβ₁₋₄₂ on B104 cells is determined by 3-(4,5-dimethylthiazol-2-yl)-2,5-diphenyltetrazolium bromide assay (In vitro Toxicology Assay Kit, MTT based, Sigma), in presence or absence of steroids. Cells are seeded in 96-well microtiter plates at the required density and grown for 24 H. They are then treated for different timings (6, 12 or 24 H) according to two procedures as mentioned above: either with specific steroids and then Aβ₁₋₄₂ or with Aβ₁₋₄₂ followed by steroids. At the end of the incubation, 10 μl of phosphate-buffered solution (PBS) containing 5 mg/ml MTT are added to each well, and the incubation is continued for another 4 H. Finally, 100 μl of a solubilization solution containing 50% dimethylformamide, 20% acidic isopropanol is added. Absorption values are determined at 590 nm by using an automatic microtiter reader. Each experiment is performed in triplicate.

### Example 7

### Trypan Blue

Trypan Blue is used to assess cell viability. B104 cells are grown in 6 wells plates for 24 H and treated under the same conditions mentioned above for flow cytometry or MTT assay. Cells are stained with 1.5% trypan blue (Sigma) solution for at least 10 min and washed with PBS. Unstained live cells are counted on a hemocytometer under a phase contrast microscope. All assays and cell counting are done in triplicate.

### Example 8

### Immunocytochemistry

The neurotrophic effects of PREGS and ent-PREGS are studied by means of immuno-labeling using certain neuronal markers: mouse anti-68 KDa neurofilament protein fluorescent antibody (Sigma), mouse anti-neuronal nuclei (NeuN) monoclonal antibody (Chemicon). For this purpose, B104 cells cultured in presence or absence (control) of specific neurosteroids for 2 to 3 days are fixed for 4 min in ethanol/acetic acid (95:5), washed and incubated for 30 min in PBS containing 2% bovine serum albumin (BSA) to block non specific binding. Immunostaining of B104 cells is performed for 2 H at room temperature (RT) with appropriate dilutions of primary antibodies. Then, cells are rinsed and incubated with sheep anti-mouse IgG antibodies (Sigma) in PBS/BSA, for 2 H at RT. After being washed, the immunostained cells are examined under microscope (a Leica or Zeiss Axiovert 135M microequipped with an epifluorescence system or optical microscope Nikon Labophot 2).

### Statistical Analysis

Data is expressed as the mean±S.E.M of three or more independent experiments, each performed in triplicates. For all experiments, control groups and treated groups are statistically compared using analysis of variance (ANOVA; for independent series with different parameters in each group), followed, when necessary by the Newman-Keuls post-hoc test. The significance level is established at p<0.05.

We claim:

1. A method for treating a subject having a neurologic disease or dysfunction comprising:
administering to the subject pregnenolone sulfate (PREGS) in an amount effective to treat the neurologic disease or dysfunction.

2. The method of claim 1, wherein the pregnenolone sulfate is the synthetic (−) enantiomer of PREGS (ent-PREGS).

3. The method of claim 1, wherein the neurologic disease or dysfunction is age-related neurodegeneration, Alzheimer's disease, Amyotrophic lateral sclerosis, Alper's diseases, Batten disease, Bovine spongiform encephalopathy (BSE), chemotherapy-induced neuropathy, Creutzfeldt-Jakob disease, diabetic neuropathy, Down's syndrome, frontotemporal lobar degeneration, Huntington's disease, HIV-associated dementia, Krabbe's disease, Lewy body dementia, multiple system atrophy, Niemann Pick disease, Parkinson's disease, Polyneuritis, Prion disease, Primary lateral sclerosis, Refsum's disease, Sandhoffs disease, senile dementia, Spielmeyer-Vogt-Sjogren-Batten disease, Spinocerebellar ataxia, Spinal muscular atrophy, Subacute combined degeneration of spinal cord, Tabes dorsalis, or Vascular dementia.

4. The method of claim 1, wherein the neurologic disease or dysfunction is caused by a burn, traumatic injury, mechanical injury, surgical injury, physiological injury, pathological injury or immunological injury.

5. The method of claim 1, wherein the neurologic disease or dysfunction is a neurodegenerative disease.

6. The method of claim 5, wherein the neurodegenerative disease is caused by the accumulation of β-amyloid peptide in neuronal tissue.

7. The method of claim 6, wherein the β-amyloid peptide is Aβ₁₄₂.

8. A method for treating a subject having a condition characterized by β-amyloid peptide accumulation in neuronal tissue comprising:
administering to the subject pregnenolone sulfate (PREGS) in an amount effective to treat the condition.

9. A method for reducing or preventing the accumulation of β-amyloid peptide in neuronal tissue comprising:
contacting the neuronal tissue with pregnenolone sulfate (PREGS) in an amount effective to reduce or prevent the accumulation of the β-amyloid peptide in the neuronal tissue.

10. The method of claim 8, wherein the β-amyloid peptide is Aβ₁₄₂.

11. The method of claim 8, wherein the pregnenolone sulfate is the synthetic (−) enantiomer of PREGS (ent-PREGS).

12. The method of claim 8, wherein the condition is Alzheimer's disease, Down's syndrome or Lewy body dementia.

13. A method for stimulating or promoting growth of a neuronal cell comprising:
contacting the neuronal cell with pregnenolone sulfate (PREGS) in an amount effective to stimulate or promote neuronal cell growth.

14. The method of claim 13, wherein the pregnenolone sulfate is the synthetic (−) enantiomer of PREGS (ent-PREGS).

15. The method of claim 13, wherein the neuronal cell is in a tissue.

16. The method of claim 15, wherein the tissue is brain tissue, spinal cord tissue or peripheral nerve tissue.

17. The method of claim 13, wherein the neuronal growth comprises neuronal outgrowth.

18. The method of claim 1, wherein the pregnenolone sulfate is administered orally, intravenously, intramuscularly, intrathecally, sublingually, buccally, intranasally, intra-articularly, intraperitoneally, subcutaneously, or topically.

19. The method of claim 1, wherein the pregnenolone sulfate is administered prophylactically.

20. The method of claim 1, wherein the subject is otherwise free of indications for treatment with pregnenolone sulfate.

21. The method of claim 1, further comprising administering an agent other than pregnenolone sulfate.
